(19) United States
(12) Patent Application Publication
(10) Pub. No.: US 2016/0175349 A1
(43) Pub. Date: Jun. 23, 2016

(54) BRAIN FUNCTION-IMPROVING COMPOSITION

(71) Applicant: Toru Hasegawa, Saga (JP)

(72) Inventor: Toru Hasegawa, Saga (JP)

(21) Appl. No.: 14/907,471

(22) PCT Filed: Jul. 30, 2014
(86) PCT No.: PCT/JP2014/070134

§ 371 Date: Jan. 25, 2016

(30) Foreign Application Priority Data
Jul. 30, 2013 (JP) 2013-157324
May 13, 2014 (JP) 2014-099609

(51) Int. Cl. A61K 33/06; A61K 31/375; A61K 36/232; A61K 36/82; A61K 36/185; A61K 33/00; A61K 31/192; A61K 31/01

(52) U.S. Cl. CPC A61K 33/06; A61K 31/192; A61K 31/375; A61K 31/01; A61K 36/82; A61K 36/185; A61K 33/00; A61K 36/232; A23L 1/30; A23L 1/302; A23L 1/3002; A23V 2002/00

(57) ABSTRACT

Provided is a brain function-improving composition which can inhibit binding of homocysteic acid to a receptor (NMDA receptor) and, at the same time, decrease the binding ability of homocysteic acid per se to the receptor to thereby improve reduction in the brain function caused by the toxicity of homocysteic acid. The brain function-improving composition comprises 0.3-0.5 part by weight of a cinnamic acid derivative compound and 0.5-0.7 part by weight of a calcium hydride powder. Also, the brain function-improving composition is characterized by further comprising 0.4-0.6 part by weight of a green tea powder, 0.4-0.6 part by weight of a cocoa powder, 0.25-0.35 part by weight of a hydroxysilica powder, and 0.9-1.1 parts by weight of a dry Angelica shikokiana Makino powder.

### CROSS-REFERENCE TO RELATED APPLICATIONS

The present application claims priority to International Application No. PCT/JP2014/070134 filed on Jul. 30, 2014, which claims priority to Japanese Patent Application No. 2013-157324 filed Jul. 30, 2013 and Japanese Patent Application No. 2014-099609 filed May 13, 2014.

### STATEMENT RE: FEDERALLY SPONSORED RESEARCH/DEVELOPMENT

Not Applicable

### BACKGROUND OF THE INVENTION

1. Technical Field

The present invention relates to a brain function-improving composition.

2. Background Art

With the arrival of recent aging society, reduction in the brain function caused by brain degenerative disease of the elderly, especially by Alzheimer's disease, Lewy body dementia, Parkinson's disease and so on becomes wide-spread problem.

If a person is attacked by such brain degenerative disease and suffers from dementia, sometimes it will even cause serious event that threatens one's dignity; therefore, great effort has been put into research on prevention and treatment of dementia in recent years.

However, there is still many undetermined parts in the causes of dementia, and development on treating method and prevention method has not been established, let alone development on corresponding medicine.

Under such circumstance, brain function-improving composition that can prevent or improve these diseases by being taken in during daily dietary life is proposed (for example, see Patent Literature 1).

According to medicine or food and drink containing such brain function-improving composition, people can daily take it in safely and easily, and can effectively improve cranial nerve disease such as dementia represented by Alzheimer's disease.

Japanese Unexamined Patent Application Publication: No. 2002-541194

However, for the above-mentioned conventional ferulic acid containing composition, there is still room for improvement on its effect.

### SUMMARY OF INVENTION

According to the inventor of the present invention, who has been doing research on nerve degenerative disease for years, in this conventional composition, ferulic acid achieves only a little brain function-improving effect by antagonizing with homocysteic acid which brings toxicity to nerve cells because of its binding to an NMD Areceptor.

However, the above-mentioned conventional ferulic acid containing composition does not deprive internal homocysteic acid per se with nerve cell toxicity of its binding ability to the receptor, so it is still hard to say that it achieves sufficient brain function-improving effect.

In view of such situation, the present invention provides a brain function-improving composition which can inhibit binding of homocysteic acid to a receptor (NMDA receptor) and, at the same time, decrease the binding ability of homocysteic acid per se to the receptor to thereby improve reduction in the brain function caused by the toxicity of homocysteic acid.

Also, in the present invention, supplementary food or brain function-improving agent which contains said brain function-improving composition is provided.

In order to solve the above-mentioned problem, the brain function-improving composition according to the present invention comprises 0.3-0.5 part by weight of a cinnamic acid derivative compound and 0.5-0.7 part by weight of a calcium hydride.

Also, the brain function-improving composition according to the present invention is characterized by comprising 0.3-0.5 part by weight of an ascorbic acid and/or 0.3-0.5 part by weight of a lycopene.

Also, the brain function-improving composition according to the present invention is characterized by further comprising 0.4-0.6 part by weight of a green tea powder, 0.4-0.6 part by weight of a cocoa powder, 0.25-0.35 part by weight of a hydroxysilica powder, and 0.9-1.1 parts by weight of a dry Angelica shikokiana Makino powder.

Also, the brain function-improving composition according to the present invention is characterized in that said cinnamic acid derivative compound is any one selected from ferulic acid, caffeic acid, sinapoic acid or the mixture thereof.

Also, the supplementary food according to the present invention is characterized by comprising said brain function-improving composition.

Also, the brain function-improving agent according to the present invention is characterized by comprising said brain function-improving composition.

The brain function-improving composition according to the present invention comprises 0.3-0.5 part by weight of the cinnamic acid derivative compound and 0.5-0.7 part by weight of the calcium hydride powder, so brain function-improving composition can be provided which can inhibit binding of homocysteic acid to the receptor (NMDA receptor) and, at the same time, decrease the binding ability of homocysteic acid per se to the receptor to thereby improve reduction in the brain function caused by the toxicity of homocysteic acid.

Also, the brain function-improving composition according to the present invention comprises 0.3-0.5 part by weight of the ascorbic acid and/or 0.3-0.5 part by weight of lycopene, so it is able to further inhibit binding of homocysteic acid to the receptor by the cinnamic acid derivative compound and the calcium hydride powder.

Also, the brain function-improving composition according to the present invention further comprises 0.4-0.6 part by weight of the green tea powder, 0.4-0.6 part by weight of the cocoa powder, 0.25-0.35 part by weight of the hydroxysilica powder, and 0.9-1.1 parts by weight of dry Angelica shikokiana Makino powder, so it has a new taste; therefore, if the medicine is to be taken daily, new choice can be provided when choosing a brain function-improving composition that agrees with the patient, and brain function-improving effect can be promoted.

Also, in the brain function-improving composition according to the present invention, said cinnamic acid derivative compound is any one selected from ferulic acid, caffeic acid, sinapoic acid or the mixture thereof, so more reliable brain function-improving effect can be achieved.

Also, the supplementary food of the present invention comprises said brain function-improving composition, so supplementary food can be provided which can inhibit binding of homocysteic acid to the receptor (NMDA receptor) and, at the same time, decrease the binding ability of homocysteic acid per se to the receptor to thereby improve reduction in the brain function caused by the toxicity of homocysteic acid.

Also, the brain function-improving agent according to the present invention comprises said brain function-improving composition, so brain function-improving agent can be provided which can inhibit binding of homocysteic acid to the receptor (NMDA receptor) and, at the same time, decrease the binding ability of homocysteic acid per se to the receptor to thereby improve reduction in the brain function caused by the toxicity of homocysteic acid.

### BRIEF DESCRIPTION OF THE DRAWINGS

FIG. 1 is an explanatory drawing showing an effect confirmation result of a brain function-improving composition according to the present embodiment.

FIG. 2 is an explanatory drawing showing the effect confirmation result of the brain function-improving composition according to the present embodiment.

FIG. 3 is an explanatory drawing showing data of the tests.

FIG. 4 is an explanatory drawing showing the effect confirmation result of the brain function-improving composition according to the present embodiment.

### DETAILED DESCRIPTION OF THE INVENTION

The present invention provides a brain function-improving composition, and provides a brain function-improving composition which comprises 0.3-0.5 part by weight of cinnamic acid derivative compound and 0.5-0.7 part by weight of calcium hydride powder.

The inventor of the present invention presumes that one pathogenetic mechanism of nerve degenerative diseases such as Alzheimer's disease, Lewy body dementia, Parkinson's disease is the excessive binding to the NMDA receptor along with increase in homocysteic acid concentration.

That is, excessive homocysteic acid inside the body binds to the NMDA receptor and excites nerve cells, nerve function is hindered, amyloid protein accumulates in nerve cells, and nerve cells are degenerated or become dead.

It is regarded that said conventional ferulic acid containing composition used to prevent or treat dementia achieves dementia prevention or treatment effect in the way that it inhibits binding of homocysteic acid to the NMDA receptor by antagonization of ferulic acid with homocysteic acid and binding of ferulic acid to the NMDA receptor.

However, though this conventional composition can use ferulic acid to antagonize with homocysteic acid to thereby inhibit binding of homocysteic acid to the receptor, it cannot decrease the binding ability of homocysteic acid per se to the receptor.

The brain function-improving composition according to the present embodiment is focused on this aspect, and comprises calcium hydride to decrease the binding ability of homocysteic acid per se to the receptor.

The calcium hydride functions in the following way: it reacts with sulfo group of homocysteic acid in, for example, peripheral blood and becomes a substance such as norvaline whose sulfo group part is modified or falls off (referred to as sulfo group modifying substance hereinafter) to thereby promote decreasing of the binding ability to the NMDA receptor.

Though only hypothetically speaking, it is considered that calcium hydride is taken in, and deoxidizes water molecules to generate active hydrogen when calcium is ionized by water inside human body, the active hydrogen reacting with the sulfo group of homocysteic acid and turning into sulfo group modifying substance.

Also, it is considered that maybe electrons released from calcium atoms during ionization react with the sulfo group of homocysteic acid which is strongly electron-withdrawing, and thereby turns into a substance whose binding ability to the NMDA receptor is lower than that of homocysteic acid.

In this way, the brain function-improving composition according to the present embodiment improves brain function by making cinnamic acid derivative compound antagonize with homocysteic acid during binding to the NMDA receptor and at the same time turning highly nerve-toxic homocysteic acid into a substance whose binding ability to the NMDA receptor is low.

Calcium hydride is not restricted as long as it can be taken in by human or other animals, among which food material is especially preferable. This kind of calcium hydride can take coral calcium powder as a typical example.

Coral calcium powder comprises about 5% of calcium hydride in the powder, and can functions as a decomposition-promoting substance which promotes the decomposition of homocysteic acid in blood.

Coral calcium powder can not only increase the dispersity of cinnamic acid derivative compound below when the brain function-improving composition according to the present embodiment is taken in, but also decompose the sulfate group of homocysteic acid in blood to inhibit binding to said receptor by active hydrogen generated by calcium hydride contained in the coral calcium powder.

Also, about the content of calcium hydride powder in the brain function-improving composition according to the present embodiment, when the content of cinnamic acid derivative compound is 0.3-0.5 part by weight, it is preferable that the content of calcium hydride powder is 0.5-0.7 part by weight. If the content of calcium hydride powder is less than 0.5 part by weight, homocysteic acid cannot be efficiently decomposed; also, if the content is more than 0.7 part by weight, there is possibility that it will taste awful, making the content unfavorable. By comprising 0.5-0.7 part by weight of calcium hydride powder, brain function-improving composition can be made which tastes good and can efficiently decompose homocysteic acid. For example, when said coral calcium is used to ensure calcium hydride content in the brain function-improving composition, by adding 6-10 parts by weight of coral calcium, calcium hydride from coral calcium in the brain function-improving composition can be 0.5-0.7 part by weight.

Also, if calcium hydride addition is further discussed, it may be about 1.5 times as much as the cinnamic acid derivative compound added to the brain function-improving composition. For example, when 0.3-0.5 part by weight of cinnamic acid derivative compound is added, it is preferable that calcium hydride powder is 0.5-0.7 part by weight. If it is less than 0.5 part by weight, there is danger that the converting ability of homocysteic acid to other substances will decrease; therefore it is unfavorable. Also, even if it is more than 0.7 part by weight, it will contribute little to improving the converting ability of homocysteic acid to other substances.

Also, the brain function-improving composition according to the present embodiment comprises 0.3-0.5 part by weight of cinnamic acid derivative compound. The cinnamic acid derivative compound in the specification is, for example, compound represented by the general formula below.

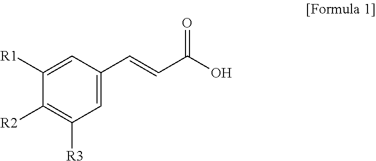

wherein R1, R2, R3 are independently any of H, OH, CH₃O

With further restriction, cinnamic acid derivative compound may be, for example, any one selected from ferulic acid, caffeic acid, sinapoic acid or the mixture thereof.

Ferulic acid is a substance functioning as a binding inhibiting substance which inhibits binding of homocysteic acid to the homocysteic acid receptor on the surface of intracerebral cells, more specifically speaking, is a substance which has a structure as represented by Formula 2 below.

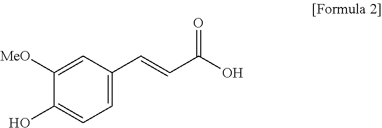 [SMILES: COc1cc(/C=C/C(=O)O)ccc1O]

The inventor of the present invention has done research for years and has found that homocysteic acid causes reduction in the brain function such as memory disorder by damaging intracerebral cells via NMDA receptor and decreasing or destroying cell function. Ferulic acid antagonizes and inhibits binding of homocysteic acid to the NMDA receptor to thereby prevent cell damage.

In addition, cinnamic acid derivative compound may be, for example, caffeic acid which has a structure as represented by Formula 3. Caffeic acid is also a substance functioning as a binding inhibiting substance which inhibits binding of homocysteic acid to the homocysteic acid receptor on the surface of intracerebral cells.

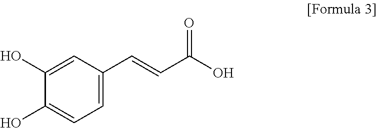 [SMILES: O=C(O)/C=C/c1ccc(O)c(O)c1]

In addition, cinnamic acid derivative compound may be, for example, sinapoic acid which has a structure as represented by Formula 4. Sinapoic acid is also a substance functioning as a binding inhibiting substance which inhibits binding of homocysteic acid to the homocysteic acid receptor on the surface of intracerebral cells.

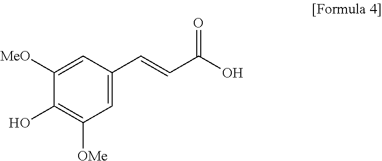 [SMILES: COc1cc(/C=C/C(=O)O)cc(OC)c1O]

In this way, the brain function-improving composition according to the present embodiment comprises cinnamic acid derivative compound whose quantity is about ⅔ as much as calcium hydride, i.e. 0.3-0.5 part by weight of cinnamic acid derivative compound; therefore, it can efficiently antagonize with homocysteic acid to improve brain function.

Besides, the brain function-improving composition according to the present embodiment may comprise 0.3-0.5 part by weight of ascorbic acid and/or 0.3-0.5 part by weight of lycopene. Ascorbic acid or lycopene can encourage the effect of active hydrogen which generates sulfo group modifying substance, and can inhibit binding of homocysteic acid to the receptor more effectively.

According to the research of inventors of the present invention, it is understood that comparing with brain function-improving composition without ascorbic acid or lycopene, the brain function-improving composition comprising these components increases generation efficiency of sulfo group modifying substance to 7-10 times.

Also, the brain function-improving composition according to the present embodiment may further comprises 0.4-0.6 part by weight of green tea powder, 0.4-0.6 part by weight of cocoa powder, 0.25-0.35 part by weight of hydroxysilica powder, and 0.9-1.1 parts by weight of dry Angelica shikokiana Makino powder.

Green tea powder is a substance functioning as a release promoting substance which promotes homocysteic acid to release out of body.

According to study of the inventor of the present invention, it is understood that the higher concentration of homocysteic acid released into urine, the lower concentration of homocysteic acid in blood, at the same time, the patient gets a higher MMSE score; by promoting homocysteic acid to release out of body, brain function is improved.

Green tea powder also functions as a masking auxiliary which masks the unique taste of cinnamic acid derivative compound along with the cocoa powder below.

As for the content of green tea powder in the brain function-improving composition, when the content of cinnamic acid derivative compound is 0.3-0.5 part by weight, it is preferably 0.4-0.6 part by weight. If it is less than 0.4 part by weight, no obvious diuretic effect can be observed and there is possibility that homocysteic acid has not been efficiently released out of body. Masking effect on cinnamic acid derivative compound also decreases, and the brain function-improving composition per se may be hard to swallow. On the other hand, even if it is more than 0.6 part by weight, the taste of green tea takes precedence and its balance with aforementioned cocoa powder is broken, masking effect on cinnamic acid derivative compound decreases. By comprising 0.4-0.6 part by weight of green tea powder, brain function-improving composition can be made which promotes efficient release of homocysteic acid and is easy to swallow.

Cocoa powder is a substance functioning as a metabolism accelerating substance which accelerates the metabolism of homocysteic acid. Specifically speaking, pyrroloquinoline quinone (PQQ) contained in cocoa powder activates glutamic acid dehydrogenase related to the metabolism of homocysteic acid and metabolism is thus accelerated.

The content of cocoa powder in the brain function-improving composition is preferably 0.4-0.6 part by weight when the content of cinnamic acid derivative compound is 0.3-0.5 part by weight. If it is less than 0.4 part by weight, no obvious metabolism accelerating effect is observed, and there is possibility that homocysteic acid has not been efficiently metabolized. Masking effect on cinnamic acid derivative compound also decreases, and the brain function-improving composition per se may be hard to swallow. On the other hand, even if it is more than 0.6 part by weight, the taste of cocoa takes precedence and its balance with abovementioned green tea powder is broken, masking effect on cinnamic acid derivative compound decreases. By comprising 0.8-1.2 parts by weight of cocoa powder, brain function-improving composition can be made which accelerates efficient metabolism of homocysteic acid and is easy to swallow.

Hydroxysilica is a substance which increases active hydrogen in peripheral blood and turns homocysteic acid into sulfo group modifying substance more efficiently.

Hydroxysilica has a structure which occludes hydrogen molecule in its crystal lattice, and has the ability to release hydrogen in peripheral blood. Released hydrogen also reacts with the sulfo group of homocysteic acid in peripheral blood, turns homocysteic acid into a sulfo group modifying substance to thereby decrease excitation effect of the NMDA receptor.

It is preferable that the content of hydroxysilica in the brain function-improving composition is about half of the calcium hydride powder content t1 (about t1×½×0.8−t1×½×1.2), that is, in a brain function-improving composition with 0.5-0.7 part by weight of calcium hydride powder, it is preferable to add 0.25-0.35 part by weight of hydroxysilica powder.

If the content of hydroxysilica is less than the calcium hydride powder content t1×½×0.8 (part by weight), efficiency of turning homocysteic acid into sulfo group modifying substance will decrease; therefore it is unfavorable. Besides, even if it is more than the calcium hydride powder content t1×½×0.8 (part by weight), efficiency of turning homocysteic acid into sulfo group modifying substance shows little increase and it is not cost-effective. By making the content of hydroxysilica fall within the range of calcium hydride powder content t1×½×0.8 (part by weight)-t1×½×0.8 (part by weight), preferably by adding 0.25-0.35 part by weight of hydroxysilica powder to a brain function-improving composition with 0.5-0.7 part by weight of calcium hydride powder, it is cost-effective and can further increase efficiency of turning homocysteic acid into sulfo group modifying substance.

Dry Angelica shikokiana Makino powder is dry powder of a plant also called Angelica acutiloba, and is used to relieve the sedative effect secondarily generated by the cinnamic acid derivative compound.

Because the cinnamic acid derivative compound is relatively sedative, people taking it sometimes falls asleep. The sedative effect may become a problem time and again in daily use.

Therefore, by comprising dry Angelica shikokiana Makino powder, the brain function-improving composition according to the present embodiment can relieve the sedative effect generated by the cinnamic acid derivative compound and people will not worry about its daily use.

It is preferable that dry Angelica shikokiana Makino powder is 0.9-1.1 part by weight in the brain function-improving composition. If it is less than 0.9 part by weight, it will be unfavorable because there is possibility that the sedative effect caused by the cinnamic acid derivative compound is not fully relieved. Besides, even if it is more than 1.1 parts by weight, sedative effect will not be further relieved, while the manufacturing cost of the brain function-improving composition will increase; therefore it is unfavorable. By comprising 0.9-1.1 part by weight of dry Angelica shikokiana Makino powder in the brain function-improving composition, brain function-improving composition can be made which can control cost and fully relieve sedative effect caused by the cinnamic acid derivative compound.

In this way, the brain function-improving composition according to the present embodiment has the composition as mentioned before, so brain function-improving composition can be provided which has a new taste and can improve brain function.

The brain function-improving composition can also be used as an ingredient for supplementary food or an ingredient for brain function-improving agent.

The brain function-improving composition according to the present embodiment may be powder or a tablet. All of said raw materials are powder, and when brain function-improving composition, supplementary food or brain function-improving agent is to be made, it may be produced by mixing the raw material powder to get a powder product or by tablet forming.

As described above, the brain function-improving composition according to the present embodiment comprises 0.3-0.5 part by weight of cinnamic acid derivative compound and 0.5-0.7 part by weight of calcium hydride powder, so brain function-improving composition can be provided which can inhibit binding of homocysteic acid to the receptor (the NMDA receptor) and, at the same time, decrease the binding ability of homocysteic acid per se to the receptor to thereby improve reduction in the brain function caused by the toxicity of homocysteic acid.

In addition, the brain function-improving composition according to the present embodiment not only can improve brain function, but also can inhibit cancer cell proliferation. That is, said brain function-improving composition can also be used as a cancer cell proliferation inhibiting composition.

According to study of the inventor of the present invention, it is discovered that homocysteic acid accelerates cancer cell proliferation by binding to the NMDA receptor of cancer cells.

However, though various compositions which inhibit cancer cell proliferation have been proposed, no composition has been proposed which can inhibit acceleration of cancer cell proliferation caused by high concentration of homocysteic acid in peripheral blood.

According to the cancer cell proliferation inhibiting composition with the same ingredients as the brain function-improving composition, cancer cell proliferation can be efficiently inhibited and chance of cancer treatment is ensured.

In the following part, the brain function-improving composition according to the present embodiment is specifically described with reference to experimental examples.

[1. Preparation of the Brain Function-Improving Composition and Verification Test (1)]

First, prepare the brain function-improving composition. Here, it is prepared in the form of supplementary food comprising the function-improving composition for the use in the aftermentioned administration test.

Prepared brain function-improving compositions include brain function-improving composition A1 comprising ferulic acid as a cinnamic acid derivative compound and calcium hydride, brain function-improving composition A2 comprising caffeic acid as a cinnamic acid derivative compound and calcium hydride, and brain function-improving composition A3 comprising sinapoic acid as a cinnamic acid derivative compound and calcium hydride. Also prepared as comparative examples are comparative composition P1 comprising more than specific content of ferulic acid as a cinnamic acid derivative compound and calcium hydride, and comparative composition P2 comprising more than specific content of ferulic acid as a cinnamic acid derivative compound and calcium hydride.

Referring to specific preparation of each composition, as to brain function-improving composition A1, 44.860 g of excipient was added to 5.607 g of ferulic acid, equal to 0.3 part by weight, and 149.533 g of coral calcium powder, equal to 8 parts by weight, and mixed in powder form to obtain 200 g of powder mixture for A1. Next, the powder mixture for A1 was sealed into hard capsules every 200 mg to make brain function-improving composition A1.

As to brain function-improving composition A2, 44.860 g of excipient was added to 5.607 g of caffeic acid, equal to 0.3 part by weight, and 149.533 g of coral calcium powder, equal to 8 parts by weight, and mixed in powder form to obtain 200 g of powder mixture for A2. Next, the powder mixture for A2 was sealed into hard capsules every 200 mg to make brain function-improving composition A2.

As to brain function-improving composition A3, 44.860 g of excipient was added to 5.607 g of sinapoic acid, equal to 0.3 part by weight, and 149.533 g of coral calcium powder, equal to 8 parts by weight, and mixed in powder form to obtain 200 g of powder mixture for A3. Next, the powder mixture for A3 was sealed into hard capsules every 200 mg to make brain function-improving composition A3.

As to comparative composition P1, 46.729 g of excipient was added to 3.738 g of ferulic acid, equal to 0.2 part by weight, and 149.533 g of coral calcium powder, equal to 8 parts by weight (equal to 0.6 part by weight as calcium hydride) and mixed in powder form to obtain 200 g of powder mixture for P1. Next, the powder mixture for P1 was sealed into hard capsules every 200 mg to make comparative composition P1.

As to comparative composition P2, 31.775 g of excipient was added to 18.692 g of ferulic acid, equal to 1 part by weight, and 149.533 g of coral calcium powder, equal to 8 parts by weight, and mixed in powder form to obtain 200 g of powder mixture for P2. Next, the powder mixture for P2 was sealed into hard capsules every 200 mg to make comparative composition P2.

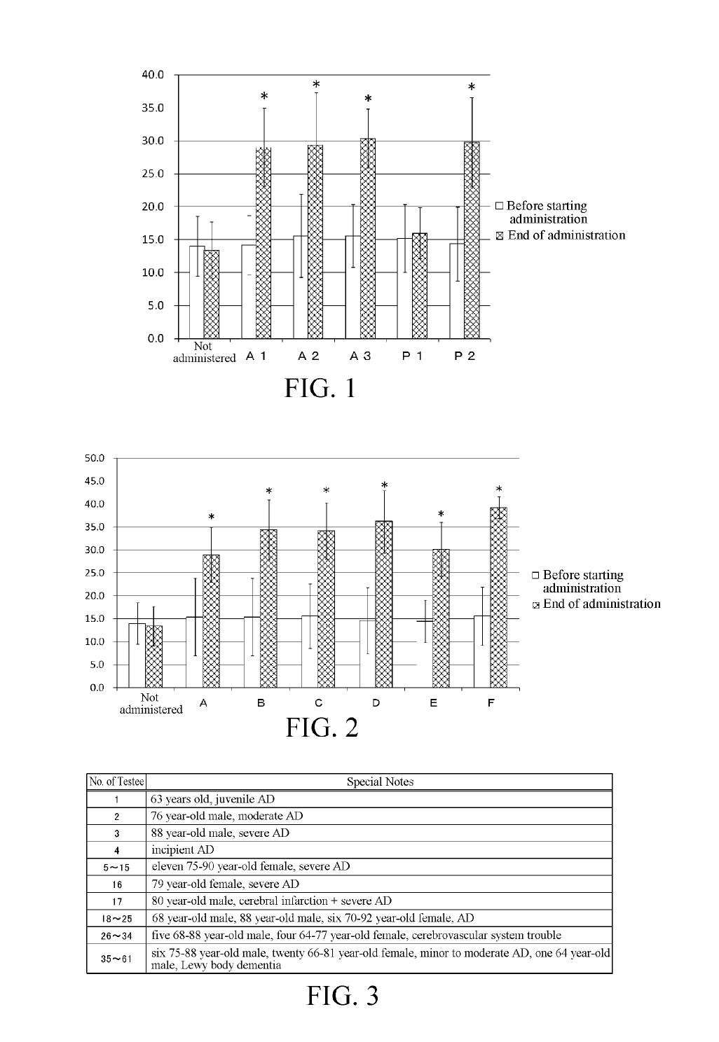

Use the brain function-improving composition A1-A3 and comparative composition P1, P2 prepared in this way and assign 5 testees for each composition to investigate brain function-improving effect. Administration of each composition was done 4 capsules each time and three times a day for 10 weeks. N-type mental state scale for the elderly (NM scale) was used for verification on improving effect. In the NM scale, 50-48 point is evaluated as a normal state, 47-43 point as a critical state, 42-31 point as minor dementia, 30-17 point as moderate dementia, 16-0 point as severe dementia. In addition, in the state before starting administration, there was no significance difference observed in NM scale evaluation value of the testees assigned for each composition. Result of the test is shown in FIG. 1.

It can be seen from FIG. 1 that both brain function-improving composition A1-A3 and comparative composition P2 showed significant increase in the evaluation point of NM scale at the end of administration compared with that before administration (P<0.01).

When focusing on the comparative composition, comparative composition P1 comprising calcium hydride and less than specific content of ferulic acid showed no significant difference in the evaluation point of NM scale before and after administration.

Also, comparative composition P2 comprising calcium hydride and more than specific content of ferulic acid showed significant difference in the evaluation point of NM scale before and after administration, but no significant difference is observed when compared with the result of brain function-improving composition A1-A3, and no significant increase in the evaluation point can be expected even with undue increase of cinnamic acid derivative compound.

From these results, it was shown that the brain function-improving composition according to the present embodiment had obvious brain function-improving effect.

[2. Preparation of the Brain Function-Improving Composition and Verification Test (2)]

Next, as for the brain function-improving composition according to the present embodiment, verification was done on effect of cases where antioxidation substance such as ascorbic acid or lycopene was added or effect of cases where green tea powder, cocoa powder, hydroxysilica powder and dry Angelica shikokiana Makino powder were added.

Specifically speaking, in the verification test, prepared are brain function-improving composition A comprising ferulic acid as a cinnamic acid derivative compound and calcium hydride, brain function-improving composition B comprising ascorbic acid in brain function-improving composition A, brain function-improving composition C comprising lycopene in brain function-improving composition A, brain function-improving composition D comprising lycopene in brain function-improving composition B, brain function-improving composition E comprising green tea powder, cocoa powder, hydroxysilica powder and dry Angelica shikokiana Makino powder in brain function-improving composition A, and brain function-improving composition F comprising green tea powder, cocoa powder, hydroxysilica powder and dry Angelica shikokiana Makino powder in brain function-improving composition B.

In the preparation of brain function-improving composition A, first, 42.990 g of excipient was added to 7.477 g of ferulic acid, equal to 0.4 part by weight, and 149.533 g of coral calcium powder, equal to 8 parts by weight and mixed in powder form to obtain 200 g of powder mixture for A. Next, the powder mixture for A was sealed into hard capsules every 200 mg to make brain function-improving composition A.

As to brain function-improving composition B, 35.513 g of excipient was added to 7.477 g of ferulic acid, equal to 0.4 part by weight, 149.533 g of coral calcium powder, equal to 8 parts by weight and 7.477 g of ascorbic acid, equal to 0.4 part by weight and mixed in powder form to obtain 200 g of powder mixture for B. Next, the powder mixture for B was sealed into hard capsules every 200 mg to make brain function-improving composition B.

As to brain function-improving composition C, 35.513 g of excipient was added to 7.477 g of ferulic acid, equal to 0.4 part by weight, 149.533 g of coral calcium powder, equal to 8 parts by weight and 7.477 g of lycopene, equal to 0.4 part by weight and mixed in powder form to obtain 200 g of powder mixture for C. Next, the powder mixture for C was sealed into hard capsules every 200 mg to make brain function-improving composition C.

As to brain function-improving composition D, 28.036 g of excipient was added to 7.477 g of ferulic acid, equal to 0.4 part by weight, 149.533 g of coral calcium powder, equal to 8 parts by weight, 7.477 g of ascorbic acid, equal to 0.4 part by weight and 7.477 g of lycopene, equal to 0.4 part by weight and mixed in powder form to obtain 200 g of powder mixture for D. Next, the powder mixture for D was sealed into hard capsules every 200 mg to make brain function-improving composition D.

As to brain function-improving composition E, mix in powder form of 7.477 g of ferulic acid, equal to 0.4 part by weight, 149.533 g of coral calcium powder, equal to 8 parts by weight, 9.346 g of green tea powder, equal to 0.5 part by weight, 9.346 g of cocoa powder, equal to 0.5 part by weight, 5.607 g of hydroxysilica powder, equal to 0.3 part by weight and 18.692 g of dry Angelica shikokiana Makino powder, equal to 1 part by weight, to obtain 200 g of powder mixture for E. Next, the powder mixture for E was sealed into hard capsules every 200 mg to make brain function-improving composition E.

As to brain function-improving composition F, mix in powder form of 7.477 g of ferulic acid, equal to 0.4 part by weight, 149.533 g of coral calcium powder, equal to 8 parts by weight, 7.477 g of ascorbic acid, equal to 0.4 part by weight, 9.346 g of green tea powder, equal to 0.5 part by weight, 9.346 g of cocoa powder, equal to 0.5 part by weight, 5.607 g of hydroxysilica powder, equal to 0.3 part by weight and 18.692 g of dry Angelica shikokiana Makino powder, equal to 1 part by weight, to obtain 207.477 g of powder mixture for E. Next, the powder mixture for E was sealed into hard capsules every 207.477 mg to make brain function-improving composition F.

Use the brain function-improving composition A-F prepared in this way, and assign 5 tests for each composition to investigate brain function-improving effect. Administration of each composition was done 4 capsules each time and three times a day for 10 weeks. N-type mental state scale for the elderly (NM scale) was used for verification on improving effect. In addition, in the state before starting administration, there was no significance difference observed in NM scale evaluation value of the tests assigned for each composition. Result of the test is shown in FIG. 2.

It can be seen from FIG. 2 that brain function-improving composition A-F showed significant increase in the evaluation point of NM scale at the end of administration compared with that before administration (P<0.01).

In addition, compared with composition A or composition E comprising no antioxidation substance, composition B-D, F with antioxidation substance such as ascorbic acid or lycopene added showed increasing tendency in evaluation point.

Composition E with green tea powder or cocoa powder, hydroxysilica powder, dry Angelica shikokiana Makino powder added showed increasing tendency in evaluation point compared with composition A comprising none of these ingredients.

From these results, it was shown that the brain function-improving composition according to the present embodiment A-F had obvious brain function-improving effect.

[3. Verification Test with Enlarged Testee Scale]

Next, use the brain function-improving composition according to the present embodiment E and enlarge testee scale to 61 testees to verify brain function-improving effect. The data of testees is shown in FIG. 3.

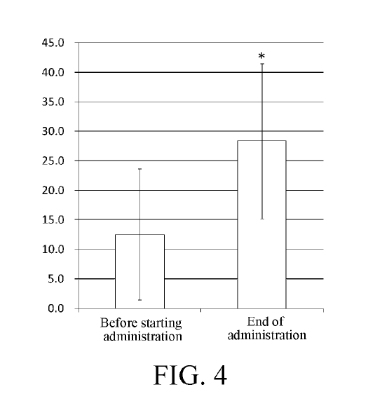

Administration of the composition was done 4 capsules each time and three times a day for 10 weeks. N-type mental state scale for the elderly (NM scale) was used for verification on improving effect. Result of the test is shown in FIG. 4.

It can also be seen from FIG. 4 that the evaluation point of NM scale of this testee group was 12.5±11.0 before administration and 28.3±13.1 at the end of administration, showing significant increase (P<0.001).

When the testee group is classified into severely ill patients (N=40) and moderately ill patients (N=21) to do statistical analysis, the evaluation point of NM scale of the severely ill patients was 5.5±4.1 before administration and 21.1±9.3 at the end of administration, showing significant increase (p<0.001). Besides, the patients classified into severely ill group according to NM scale before administration was improved to the level of moderately ill group classified according to NM scale at the end of administration.

The evaluation point of NM scale of moderately ill patients was 25.9±7.0 before administration and 42.3±6.3 at the end of administration, showing significant increase (p<0.001). That is, the patients classified into moderately ill group according to NM scale before administration was improved to the level classified as critical state or minor dementia according to NM scale at the end of administration.

It was shown from these results that the brain function-improving composition according to the present embodiment has brain function-improving effect on patients of various severities.

As described above, the brain function-improving composition according to the present embodiment comprises 0.3-0.5 part by weight of cinnamic acid derivative compound and 0.5-0.7 part by weight of calcium hydride powder, so brain function-improving composition can be provided which can improve reduction in the brain function. Supplementary food or brain function-improving agent can also be provided which comprises brain function-improving composition and is able to achieve the same effect.

At last, the description of the embodiments is only an illustration of the present invention and is not to be taken by way of limitation of the present invention. Therefore, it is clear that various changes can also be to embodiments besides the above-mentioned ones according to design, etc. as long as it is within the scope of the technical spirit of the present invention.

1. A brain function-improving composition which comprises 0.3-0.5 part by weight of cinnamic acid derivative compound and 0.5-0.7 part by weight of calcium hydride powder.

2. The brain function-improving composition according to claim 1, characterized by comprising 0.3-0.5 part by weight of ascorbic acid and/or 0.3-0.5 part by weight of lycopene.

3. The brain function-improving composition according to claim 1, characterized by further comprising 0.4-0.6 part by weight of green tea powder, 0.4-0.6 part by weight of cocoa powder, 0.25-0.35 part by weight of hydroxysilica powder, and 0.9-1.1 parts by weight of dry Angelica shikokiana Makino powder.

4. The brain function-improving composition according to claim 1, characterized in that said cinnamic acid derivative compound is any one selected from ferulic acid, caffeic acid, sinapoic acid or the mixture thereof.

5. A supplementary food, which comprises the brain function-improving composition according to claim 1.

6. A brain function-improving agent, which comprises the brain function-improving composition according to claim 1.

7. The brain function-improving composition according to claim 2, characterized by further comprising 0.4-0.6 part by weight of green tea powder, 0.4-0.6 part by weight of cocoa powder, 0.25-0.35 part by weight of hydroxysilica powder, and 0.9-1.1 parts by weight of dry Angelica shikokiana Makino powder.

8. The brain function-improving composition according to claim 2, characterized in that said cinnamic acid derivative compound is any one selected from ferulic acid, caffeic acid, sinapoic acid or the mixture thereof.

9. The brain function-improving composition according to claim 3, characterized in that said cinnamic acid derivative compound is any one selected from ferulic acid, caffeic acid, sinapoic acid or the mixture thereof.

10. A supplementary food, which comprises the brain function-improving composition according to claim 2.

11. A supplementary food, which comprises the brain function-improving composition according to claim 3.

12. A supplementary food, which comprises the brain function-improving composition according to claim 4.

13. A brain function-improving agent, which comprises the brain function-improving composition according to claim 2.

14. A brain function-improving agent, which comprises the brain function-improving composition according to claim 3.

15. A brain function-improving agent, which comprises the brain function-improving composition according to claim 4.
